(19) United States
(12) Patent Application Publication
(10) Pub. No.: US 2023/0215526 A1
(43) Pub. Date: Jul. 6, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Tokyo (JP); Nobuya KITAMURA, Tokyo (JP); Tomohide HIRAGAMI, Tokyo (JP); Yasuyuki HOSONO, Tokyo (JP); Kenji NAGAMIYA, Tokyo (JP)

(21) Appl. No.: 18/059,444

(22) Filed: Nov. 29, 2022

(30) Foreign Application Priority Data
Jan. 4, 2022 (JP) 2022-000128

(51) Int. Cl. G16H 10/60

(52) U.S. Cl. CPC G16H 10/60

(57) ABSTRACT

An information processing apparatus includes at least one processor. The processor acquires medical document data in which medical information obtained by a medical examination of a user is recorded, acquires at least one of input information which is input to a user terminal device used by the user or biological information which is measured for the user, extracts related-information which is related to the medical information from at least one of the input information or the biological information, and performs association processing of associating the extracted related-information with related medical information.

### CROSS-REFERENCE TO RELATED APPLICATIONS

The present application claims priority under 35 U.S.C. §119 to Japanese Patent Application No. 2022-000128 filed on Jan. 4, 2022. The above application is hereby expressly incorporated by reference, in its entirety, into the present application.

### BACKGROUND OF THE INVENTION

### 1. Technical Field

The disclosed technology relates to an information processing apparatus, an information processing method, and an information processing program.

### 2. Description of the Related Art

The following technology is known as technology related to information processing related to health management of a user. For example, JP2014-85986A discloses a health management device that associates pieces of individual information included in each of a first information group, which is a set of a plurality of pieces of schedule information, a second information group, which is a set of a plurality of pieces of biological information, and a third information group, which is a set of a plurality of pieces of comment information, with each other based on a match or similarity of words included in each individual information, receives selection of any individual information included in any of the first to third information groups, and displays another individual information associated with the selected individual information in an enhanced manner.

### SUMMARY

In the medical document data, such as an electronic medical record, medical information, such as a symptom obtained through an interview with a patient, is recorded. However, there is a case in which the patient is not clearly aware of his/her symptom or cannot correctly report his/her symptom to a doctor. In such a case, information lacking in the accuracy is recorded in the medical document data, and as a result, there is a risk that it is difficult to appropriately formulate an examination order, make a diagnosis, perform a treatment plan, and the like.

The disclosed technology is to contribute to the improvement of the accuracy of medical information recorded in medical document data.

The disclosed technology relates to an information processing apparatus comprising at least one processor. The processor is configured to acquire medical document data in which medical information obtained by a medical examination of a user is recorded, acquire at least one of input information which is input to a user terminal device used by the user or biological information which is measured for the user, extract related-information which is related to the medical information from at least one of the input information or the biological information, and perform association processing of associating the extracted related-information with related medical information.

According to the disclosed technology, it is possible to contribute to the acquisition of accurate medical information as compared with a case in which at least one of the input information which is input to the user terminal device or the biological information, which is measured for the user is not acquired.

### BRIEF DESCRIPTION OF THE DRAWINGS

Exemplary embodiments according to the technique of the present disclosure will be described in detail based on the following figures, wherein:

FIG. 1 is a diagram showing an example of a configuration of an information processing system according to an embodiment of the disclosed technology;

FIG. 2 is a diagram showing an example of medical information recorded in an electronic medical record according to the embodiment of the disclosed technology;

FIG. 3 is a diagram showing an example of a hardware configuration of an information processing apparatus according to the embodiment of the disclosed technology;

FIG. 4 is a functional block diagram showing an example of a functional configuration of the information processing apparatus according to the embodiment of the disclosed technology;

FIG. 5 is a diagram showing an example of a display form of the medical information recorded in the electronic medical record according to the embodiment of the disclosed technology;

FIG. 6 is a diagram showing an example of a display form of related-information according to the embodiment of the disclosed technology;

FIG. 7 is a diagram showing an example of the display form of the related-information according to the embodiment of the disclosed technology;

FIG. 8 is a flowchart showing an example of a flow of processing performed by executing an information processing program according to the embodiment of the disclosed technology;

FIG. 9 is a functional block diagram showing an example of a functional configuration of an information processing apparatus according to another embodiment of the disclosed technology; and

FIG. 10 is a diagram showing an example of a display form of a comparison result according to the embodiment of the disclosed technology.

### DETAILED DESCRIPTION

In the following, an example of embodiments of the disclosed technology will be described with reference to the drawings. It should be noted that the same or equivalent components and portions in the drawings are represented by the same reference numerals, and the overlapping description will be omitted.

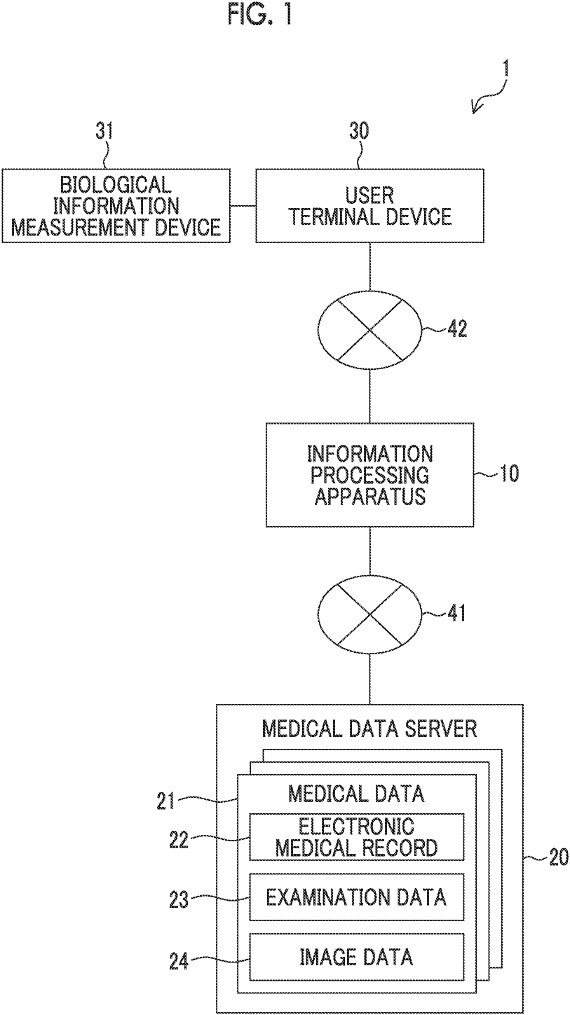

FIG. 1 is a diagram showing an example of a configuration of an information processing system 1 according to the embodiment of the disclosed technology. The information processing system 1 includes an information processing apparatus 10, a medical data server 20, a user terminal device 30, and a biological information measurement device 31.

The medical data server 20 is a computer which manages medical data of a patient. Medical data 21 for each patient is stored in the medical data server 20. The medical data 21 includes at least an electronic medical record 22. Moreover, the medical data 21 may include image data 24 and examination data 23. The electronic medical record 22 is medical document data in which medical information obtained by a medical examination of a user who is the patient is recorded. The examination data 23 is data indicating results of various examinations, such as a sample examination and a physiological function examination, performed on the patient. The image data 24 is data of various medical images, such as an X-ray image, an ultrasound image, a computed tomography (CT) image, and a magnetic resonance imaging (MRI) image captured for the patient.

The information processing apparatus 10 is communicably connected to the medical data server 20 via a network 41, and can access the medical data 21 stored in the medical data server 20. The information processing apparatus 10 can view the electronic medical record 22, the examination data 23, and the image data 24, and can create and edit the electronic medical record 22. The information processing apparatus 10 and the medical data server 20 may be installed in a medical institution, such as a hospital, and in this case, the network 41 may be a local area network (LAN) constructed in the medical institution. Moreover, the medical data server 20 may be a so-called cloud server. In this case, the network 41 may be a wide area network (WAN).

The user terminal device 30 is a computer used by the user who is the patient in which the medical data 21 is recorded in the medical data server 20. The user terminal device 30 may be, for example, a personal computer or a smartphone which is personally used by the user (patient). The user terminal device 30 can be connected to the information processing apparatus 10 via a network 42. The network 42 may be at least any one of the local area network (LAN) or the wide area network (WAN), or a combination thereof.

Various applications, such as a web browser, an e-mail application, a messenger application, and an SNS application, are installed in the user terminal device 30, and the user can use these applications. For example, the user can perform a web search by inputting a search keyword into a search engine by using the user terminal device 30. Moreover, the user can transmit or dispatch a message or information to a specified or unspecified party by using the e-mail application, the messenger application, the social networking service (SNS) application, and the like installed in the user terminal device 30. It should be noted that only one user terminal device 30 is shown in FIG. 1, but the information processing system 1 can include a plurality of user terminal devices 30.

The biological information measurement device 31 measures biological information of the user (patient). For example, the biological information may be information indicating at least one of a body temperature, a heartbeat, electrocardiography, myoelectricity, a blood pressure, arterial oxygen saturation (SpO2), a blood glucose level, or a lipid level. Moreover, the biological information may include a fluctuation rate of these measurement values in any period. The biological information measurement device 31 may be a wearable device, such as a smart watch, which comprises a sensor that measures these pieces of the biological information. The biological information measurement device 31 has a function of continuously measuring the biological information of the user in accordance with a predetermined time schedule and transmitting the measurement value to the user terminal device 30. The biological information measurement device 31 may perform measurement for various measurement items, for example, once or twice or more times daily. A measurement frequency may be different for each measurement item. Moreover, the user may be able to set any time for the time schedule of the measurement. The connection between the user terminal device 30 and the biological information measurement device 31 may be in any form of wireless or wired. Moreover, the user terminal device 30 and the biological information measurement device 31 may be connected to each other via the network configured by at least any one of the local area network (LAN) or the wide area network (WAN), or a combination thereof.

The user terminal device 30 transmits the biological information measured by the biological information measurement device 31 to the information processing apparatus 10. Moreover, the user terminal device 30 transmits input information, which is input to the user terminal device 30, to the information processing apparatus 10. The input information may be information input on various applications installed in the user terminal device 30. For example, the input information may include the search keyword input to the search engine for searching for information on the network. Moreover, the input information may include a word transmitted or dispatched to a specified or unspecified party, such as a transmission mail, the message on the messenger application, dispatch on the SNS, description of the weblog, or a chat conversation.

The user terminal device 30 may immediately transmit these pieces of information to the information processing apparatus 10 after the biological information is measured and after the input information is input, or the user terminal device 30 may transmit these pieces of information to the information processing apparatus 10 at a predetermined time point. Moreover, the user terminal device 30 may transmit the biological information and the input information to the information processing apparatus 10 based on a transmission instruction of these pieces of information. The transmission instruction of the biological information and the input information may be transmitted from the information processing apparatus 10. The information processing apparatus 10 performs information processing for supporting the creation of the electronic medical record 22, by using the biological information and the input information transmitted from the user terminal device 30. Moreover, the user may be able to set any time for a transmission timing to the information processing apparatus 10.

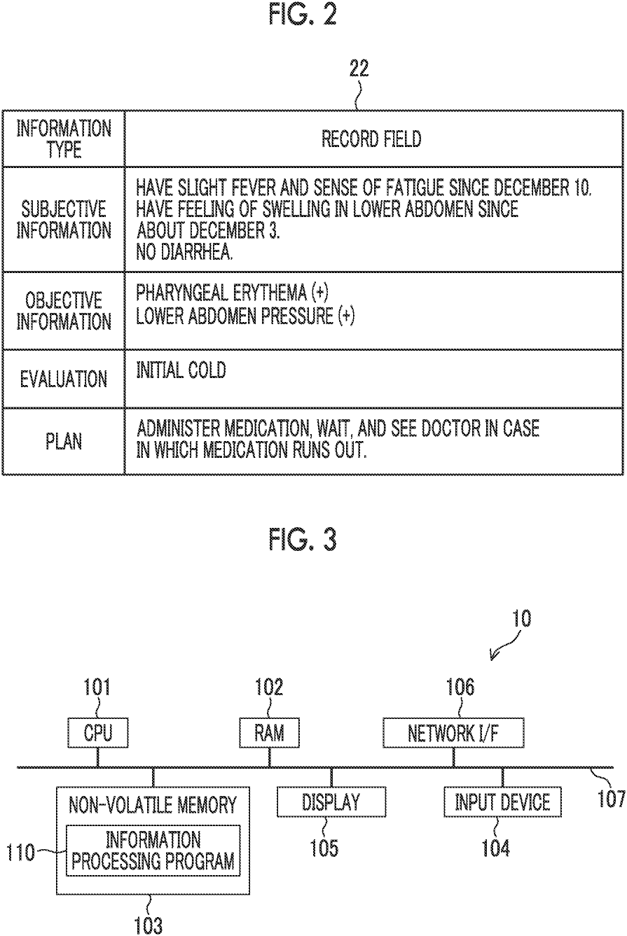

FIG. 2 is a diagram showing an example of the medical information recorded in the electronic medical record 22. The medical information recorded in the electronic medical record 22 includes “subjective information”, “objective information”, “evaluation”, and “plan”. The “subjective information” is information obtained through an interview with the patient, and examples thereof include information on a subj ective symptom, an onset time, a current medical history, a past history, a family history, and a life history complained by the patient himself/herself. The “objective information” is objective information obtained by performing the medical examination and examination on the patient. The “evaluation” is information indicating a comprehensive evaluation performed by an analysis or interpretation based on a diagnosis of a doctor, the subjective information, and the objective information. The “plan” is information indicating a treatment policy, a content, a lifestyle guide, and the like determined based on the “evaluation”. The electronic medical record 22 can be viewed, created, and edited by, for example, the doctor using the information processing apparatus 10. The created electronic medical record 22 is stored and saved in the medical data server 20.

FIG. 3 is a diagram showing an example of a hardware configuration of the information processing apparatus 10. The information processing apparatus 10 includes a central processing unit (CPU) 101, a random access memory (RAM) 102, a non-volatile memory 103, an input device 104, a display 105, and a network interface 106. These pieces of hardware are connected to a bus 107.

For example, the display 105 is a liquid crystal display or a light emitting diode (LED) display. The input device 104 includes, for example, a keyboard and a mouse, and may include a near input device, such as a touch panel display, a voice input device, such as a microphone, and a contactless input device, such as a camera or a sensor. The network interface 106 is an interface for connecting the information processing apparatus 10 to the networks 41 and 42.

The non-volatile memory 103 is a non-volatile storage medium, such as a hard disk and a flash memory. An information processing program 110 is stored in the non-volatile memory 103. The RAM 102 is a work memory for the CPU 101 to execute processing. The CPU 101 loads the information processing program 110 stored in the non-volatile memory 103 into the RAM 102, and executes the processing in accordance with the information processing program 110. The CPU 101 is an example of a “processor” according to the disclosed technology.

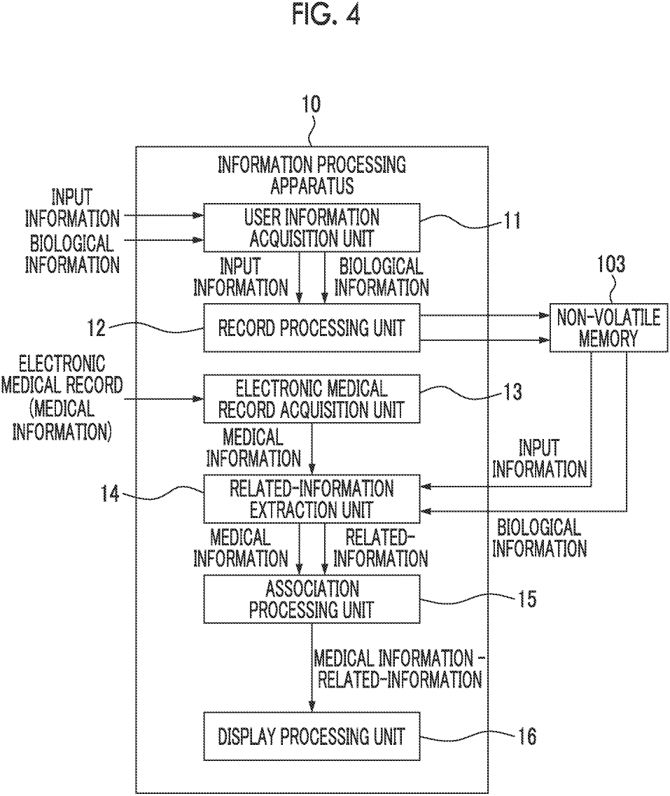

FIG. 4 is a functional block diagram showing an example of a functional configuration of the information processing apparatus 10. The information processing apparatus 10 includes a user information acquisition unit 11, a record processing unit 12, an electronic medical record acquisition unit 13, a related-information extraction unit 14, an association processing unit 15, and a display processing unit 16. By the CPU 101 executing the information processing program 110, the CPU 101 functions as the user information acquisition unit 11, the record processing unit 12, the electronic medical record acquisition unit 13, the related-information extraction unit 14, the association processing unit 15, and the display processing unit 16.

The user information acquisition unit 11 acquires the biological information and the input information of the user (patient) transmitted from the user terminal device 30. For example, the user information acquisition unit 11 acquires the biological information and the input information at a frequency of once or twice or more times a day.

The record processing unit 12 performs processing of giving metadata including date and time information indicating measurement date and time of the biological information and a user identification (ID) to the biological information acquired by the user information acquisition unit 11, and recording (saving) the biological information to which the metadata is given in the non-volatile memory 103. Moreover, the record processing unit 12 performs selection processing for selecting input information including a specific word related to a physical condition of the user (patient) out of the input information acquired by the user information acquisition unit 11. The record processing unit 12 performs processing of giving metadata including date and time information indicating input date and time of the input information and the user ID to the selected input information and recording (saving) the input information to which the metadata is given in the non-volatile memory 103.

Examples of the specific word related to the physical condition of the user (patient) include “fever”, “cough”, “headache”, “stomachache”, “dizziness”, “cold”, “sleeplessness”, “uneasiness”, “depressed”, “listless”, “annoyed”, “influenza”, “novel coronavirus”, and “COVID-19”. Such a specific word related to the physical condition of the user (patient) may be registered in advance. In this case, the record processing unit 12 may select the input information including the word registered in advance out of the input information acquired by the user information acquisition unit 11 and record the selected input information in the non-volatile memory 103. By performing the selection processing described above on the input information, the input information irrelevant to the physical condition of the user (patient) is excluded from a processing target. The biological information and the input information transmitted daily from the user terminal device 30 are accumulated in the non-volatile memory 103 of the information processing apparatus 10.

The electronic medical record acquisition unit 13 acquires the electronic medical record 22 in which the medical information is recorded. For example, the electronic medical record acquisition unit 13 may acquire the electronic medical record 22 in a case in which the creation of the electronic medical record 22 using the information processing apparatus 10 is completed. Moreover, the electronic medical record acquisition unit 13 may acquire the created electronic medical record 22 stored in the medical data server 20.

The related-information extraction unit 14 extracts the related-information which is related to the medical information recorded in the electronic medical record 22 acquired by the electronic medical record acquisition unit 13 from at least one of the biological information or the input information of the patient recorded in the non-volatile memory 103. The related-information extraction unit 14 specifies the biological information and the input information of the same patient as a target patient of the electronic medical record 22 with reference to the user ID given to the biological information and the input information, and extracts the related-information from the specified biological information and input information.

For example, the related-information extraction unit 14 may extract the input information including a description indicating a symptom that is the same as or similar to the symptom recorded in a record field of the subjective information of the electronic medical record 22 as the related-information. For example, in a case in which “slight fever” is recorded as the subjective symptom of the patient in the record field of the subjective information of the electronic medical record 22, the input information including descriptions, such as “slight fever”, “fever”, and “feverish”, may be extracted as the related-information.

Moreover, the related-information extraction unit 14 may extract the input information including a description indicating a symptom that is expected to occur in relation to the symptom recorded in the record field of the subjective information of the electronic medical record 22 as the related-information. For example, in a case in which “cough” is recorded as the subjective symptom of the patient in the record field of the subjective information of the electronic medical record 22, the input information including descriptions, such as “headache”, “nasal discharge”, “throat pain”, and “breathe with difficulty” that may occur in relation to the symptom of cough, may be extracted as the related-information.

Moreover, the related-information extraction unit 14 may extract the input information, which is input within a predetermined period before and after the onset time of the symptom recorded in the record field of the subjective information of the electronic medical record 22, and the biological information measured within the predetermined period as the related-information. For example, in a case in which the record field of the subjective information of the electronic medical record 22 includes the description “slight fever since December 10”, the input information, which is input within a period of one week before and after December 10, and the biological information measured within the period described above may be extracted as the related-information.

Moreover, the related-information extraction unit 14 may extract the input information, which is input within the predetermined period before a time when the medical information is recorded in the electronic medical record 22, and the biological information measured within the predetermined period as the related-information. For example, in a case in which the time when the medical information is recorded in the electronic medical record 22 is October 20, the input information, which is input in one week until October 20 (that is, a period from October 13 to October 20), and the biological information measured within the period described above may be extracted as the related-information.

The association processing unit 15 performs association processing of associating the related-information extracted by the related-information extraction unit 14 with the related medical information recorded in the electronic medical record 22. That is, the medical information of a certain patient recorded in the electronic medical record 22 is associated with the related-information which is related to the medical information and extracted from the input information and the biological information of the patient. The association processing unit 15 may set, as one of the association processing, a hyperlink indicating a location of the corresponding related-information in the medical information recorded in the electronic medical record 22.

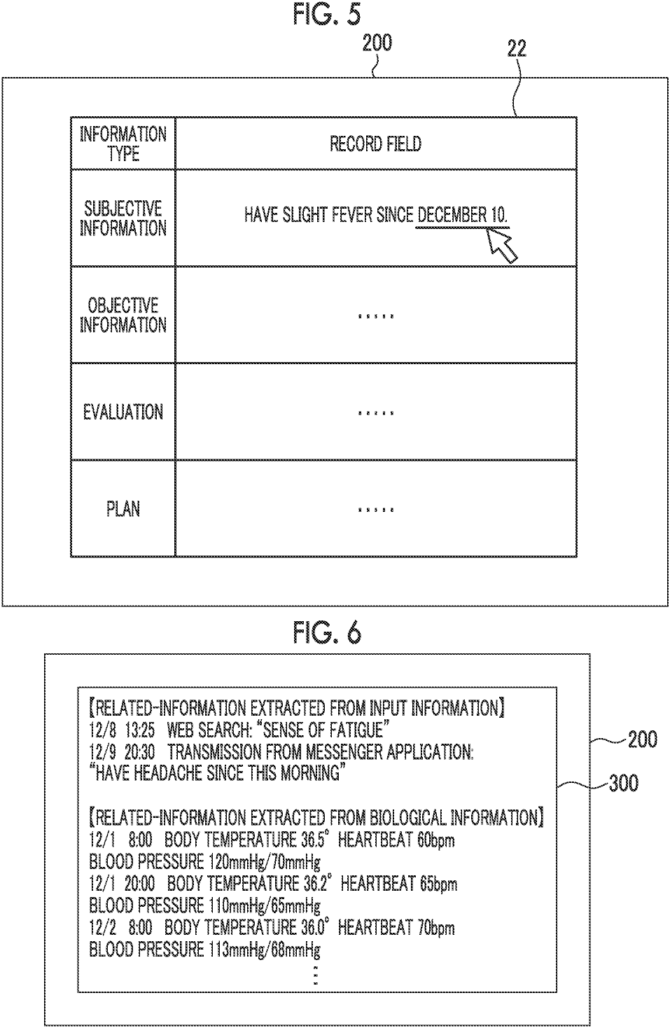

The display processing unit 16 displays the medical information recorded in the electronic medical record 22 acquired by the electronic medical record acquisition unit 13 on a display screen 200 of the display 105. FIG. 5 is a diagram showing an example of a display form of the medical information recorded in the electronic medical record 22. FIG. 5 shows a state in which the electronic medical record 22 in which the subjective information of “have a slight fever since December 10” obtained through the interview with the patient is recorded is displayed on the display screen 200. Moreover, FIG. 5 shows a case in which the extraction processing and the association processing of the related-information for the input information and the biological information are performed based on the description of “December 10” in the electronic medical record 22 and the hyperlink of the related-information is set in the description portion of “December 10” in the electronic medical record 22.

In a case in which a predetermined input operation or selection operation is performed in a state in which the medical information recorded in the electronic medical record 22 is displayed on the display screen 200, the display processing unit 16 displays the related-information associated with the medical information on the display screen 200. For example, in a case in which the input operation of clicking the description portion of “December 10” in which the hyperlink is set in the electronic medical record 22 shown in FIG. 5 is performed using the input device 104, such as the mouse, the display processing unit 16 displays the related-information associated with the medical information on the display screen 200.

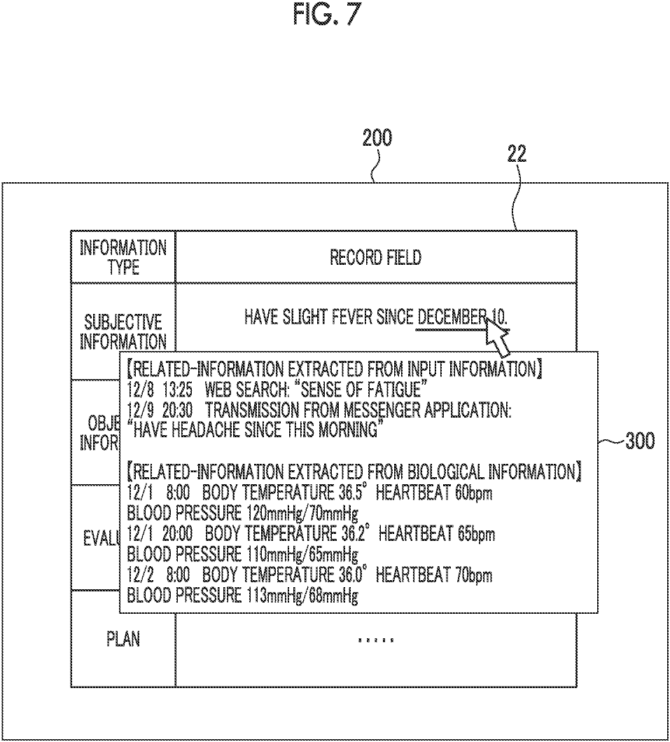

FIG. 6 is a diagram showing an example of a display form of the related-information. In FIG. 6, the input information, which is input within the predetermined period before and after “December 10” described in the record field of the subjective information of the electronic medical record 22, and the biological information measured within the period described above are shown as related-information 300. The related-information 300 may be displayed on the same screen as the screen on which the electronic medical record 22 is displayed, or may be displayed on another screen. It is preferable that the related-information 300 be displayed in a manner that can be clearly distinguished from the medical information recorded in the electronic medical record 22.

FIG. 7 is a diagram showing another example of the display form of the related-information. In the example shown in FIG. 7, in a state in which the medical information recorded in the electronic medical record 22 is displayed on the display screen 200, the related-information 300 associated with the medical information is displayed on the display screen 200 by a mouse over pop-up. For example, in a case in which the selection operation of placing a cursor on the description portion of “December 10” in which the hyperlink is set in the electronic medical record 22 shown in FIG. 7 is performed using the input device 104, such as the mouse, the display processing unit 16 displays the related-information associated with the medical information on the display screen 200 in a pop-up manner.

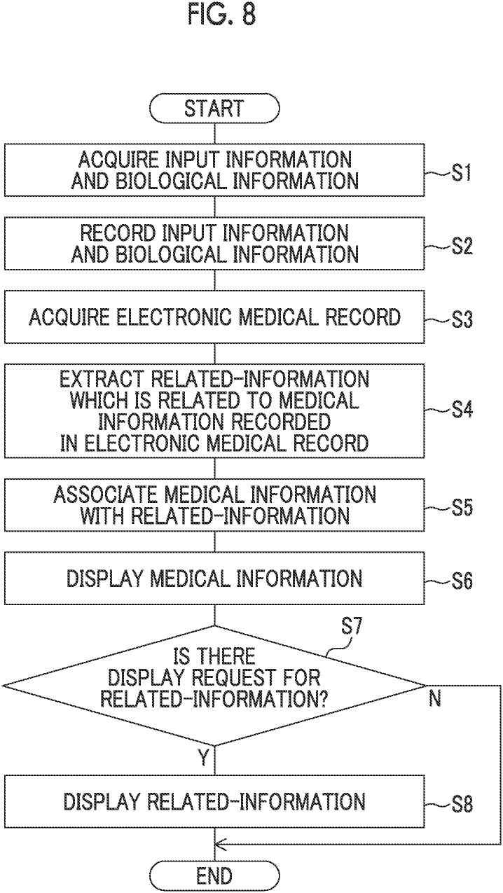

FIG. 8 is a flowchart showing an example of a flow of processing performed by the CPU 101 executing the information processing program 110. The information processing program 110 is executed, for example, in a case in which the user instructs the start of processing by operating the input device 104.

In step S1, the CPU 101 functions as the user information acquisition unit 11 to acquire the biological information and the input information of the user (patient) which are transmitted from the user terminal device 30. The CPU 101 acquires the biological information and the input information at, for example, a frequency of once or twice or more times a day.

In step S2, the CPU 101 functions as the record processing unit 12 to perform processing of giving the metadata including the date and time information indicating the measurement date and time and the user ID in the biological information acquired in step S1 and recording (saving) the biological information to which the metadata is given in the non-volatile memory 103. Moreover, the CPU 101 performs the selection processing of selecting the input information including the specific word related to the physical condition of the user (patient) out of the input information acquired in step S1 performs the processing of giving the metadata including the date and time information indicating the input date and time of the input information and the user ID to the selected input information and recording (saving) the input information to which the metadata is given in the non-volatile memory 103. The biological information and the input information transmitted daily from the user terminal device 30 are accumulated in the non-volatile memory 103 of the information processing apparatus 10.

In step S3, the CPU 101 functions as the electronic medical record acquisition unit 13 to acquire the electronic medical record 22 in which the medical information is recorded in the record field. The CPU 101 may acquire the electronic medical record 22 in a case in which the creation of the electronic medical record 22 using the information processing apparatus 10 is completed. Moreover, the CPU 101 may acquire the created electronic medical record 22 stored in the medical data server 20.

In step S4, the CPU 101 functions as the related-information extraction unit 14 to extract the related-information which is related to the medical information recorded in the electronic medical record 22 acquired in step S3 from at least one of the biological information or the input information of the patient recorded in the non-volatile memory 103 in step S2. The related-information extraction unit 14 specifies the biological information and the input information of the same patient as the target patient of the electronic medical record 22 with reference to the user ID given to the biological information and the input information, and extracts the related-information from the specified biological information and input information.

In step S5, the CPU 101 functions as the association processing unit 15 to perform the association processing of associating the related-information extracted in step S4 with the related medical information recorded in the electronic medical record 22. For example, the CPU 101 may set the hyperlink indicating the location of the corresponding related-information in the medical information recorded in the electronic medical record 22.

In step S6, the CPU 101 functions as the display processing unit 16 to display the medical information, which is recorded in the electronic medical record 22 acquired in step S3, on the display screen 200 of the display 105. In step S7, the CPU 101 determines whether or not there is a display request for the related-information. The display request for the related-information can be made, for example, by the input operation of clicking the description portion in which the hyperlink of the medical information displayed on the display screen 200 is set or the selection operation of placing the cursor on the description portion.

In a case in which it is determined that there is the display request for the related-information, the CPU 101 functions as the display processing unit 16 in step S8 to display the related-information on which the association processing is performed in step S5 on the display screen 200. In a case in which it is determined that there is no display request for the related-information, the CPU 101 terminates the present routine.

As described above, the information processing apparatus 10 according to the present embodiment acquires the electronic medical record 22 which is the medical document data in which the medical information obtained by the medical examination of the user is recorded, and acquires at least one of the input information which is input to the user terminal device 30 used by the user or the biological information which is measured for the user. The information processing apparatus 10 extracts the related-information which is related to the medical information recorded in the electronic medical record 22 from at least one of the input information or the biological information, and performs the association processing of associating the extracted related-information with the related medical information. The information processing apparatus 10 displays the medical information on the display screen 200, and displays the related-information, which is associated with the medical information displayed on the display screen 200, on the display screen 200 based on the input operation or the selection operation.

In the medical document data, such as the electronic medical record, the symptom or the like obtained through the interview with the patient is recorded. However, there is a case in which the patient is not clearly aware of his/her symptom or cannot correctly report his/her symptom to the doctor. In such a case, information lacking in the accuracy is recorded in the medical document data, such as the electronic medical record, and as a result, there is a risk that it is difficult to appropriately formulate an examination order, make a diagnosis, perform a treatment plan, and the like.

With the information processing apparatus 10 according to the embodiment of the disclosed technology, the related-information which is related to the medical information recorded in the electronic medical record 22 is associated with the medical information. The related-information is extracted from the input information which is input to the user terminal device 30 used by the user who is the target patient of the electronic medical record 22 or the biological information measured for the user. It is considered that information on the symptom or the physical condition of the user (patient) is recorded with relatively high accuracy in the input information and the biological information. By associating the related-information extracted from the input information and the biological information with the medical information recorded in the electronic medical record 22 and displaying the related-information associated with the medical information in response to the request, the authenticity of the medical information recorded in the electronic medical record 22 can be evaluated. For example, the doctor interviews the patient again in a case in which a content of the medical information recorded in the electronic medical record 22 and a content of the related-information associated with the medical information do not match, and the doctor can take measures, such as correcting medical information in a case in which the medical information recorded in the electronic medical record 22 lacks in the accuracy as a result of the determination as to whether or not the medical information is accurate.

As described above, with the information processing apparatus 10 according to the embodiment of the disclosed technology, it is possible to contribute to the improvement of the accuracy of the medical information recorded in the medical document data as compared with a case in which at least one of the input information which is input to the user terminal device or the biological information which is measured for the user is not acquired. In particular, it is effective to perform the association processing according to the present embodiment with respect to the subj ective information obtained through the interview with the user among various pieces of information recorded in the medical document data. This is because the subjective information often lacks in the accuracy.

It should be noted that, in the present embodiment, the case in which the extraction processing and the association processing of the related-information are performed on the subjective information out of the medical information recorded in the electronic medical record 22 has been described as a typical example, but the extraction processing and the association processing of the related-information may be performed on information other than the subjective information.

Moreover, in the present embodiment, the electronic medical record is described as the medical document data in which the medical information is recorded, but it is also possible to apply the disclosed technology to medical document data other than the electronic medical record.

Moreover, in the present embodiment, the case has been described in which both the input information and the biological information are acquired as user information which is an extraction source of the related-information, but only one of the input information and the biological information may be acquired to extract the related-information from the acquired input information or biological information.

Moreover, in the present embodiment, the case has been described in which the information processing apparatus 10 acquires the biological information and the input information transmitted from the user terminal device 30, but in a case in which the biological information and the input information are accumulated in a predetermined server, the information processing apparatus 10 may access the server and acquire the biological information and the input information.

The information processing apparatus 10 according to the present embodiment is an example of an “information processing apparatus” according to the technology of the present disclosure. Moreover, the “information processing apparatus” according to the technology of the present disclosure may be a system including the configurations of the information processing apparatus 10 and the medical data server 20. More specifically, a system including the medical data server 20 and the information processing apparatus 10 which include at least the electronic medical record 22 may be used as the information processing apparatus. Moreover, in the present embodiment, the acquisition of the user information is periodically performed at a frequency of once or twice or more times a day, but the disclosed technology is not limited to this. For example, the user information acquisition unit 11 may acquire the user information by using a timing of accessing the user information in the electronic medical record or starting the input of the electronic medical record as a trigger in a case in which the user visits the hospital.

### Second Embodiment

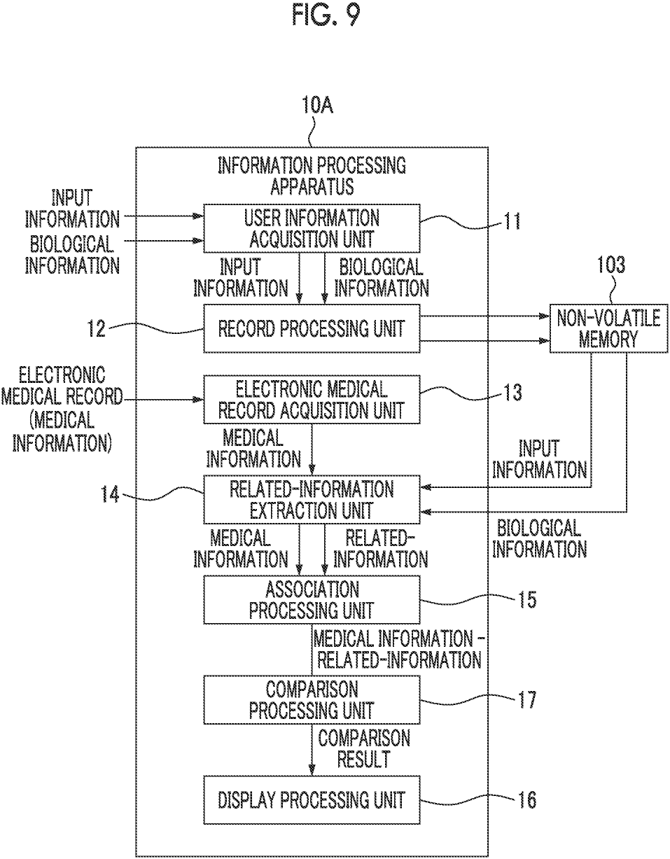

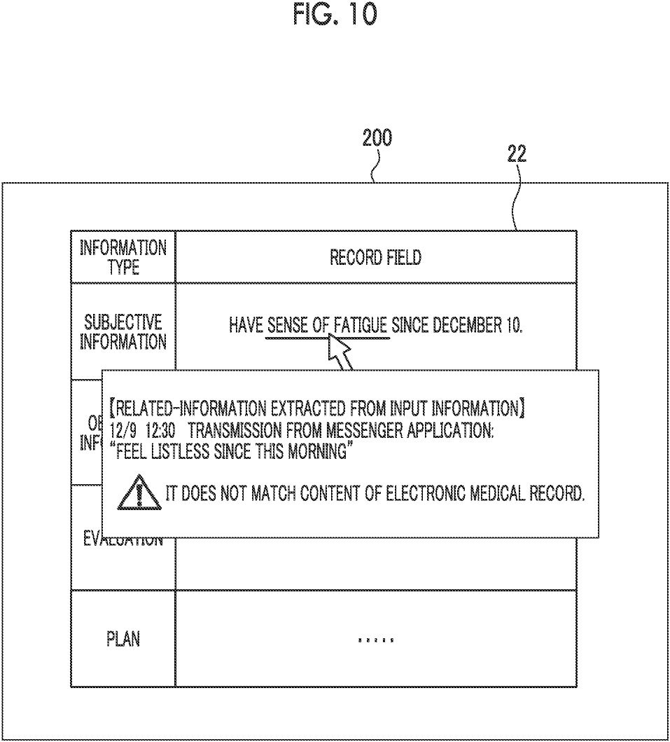

FIG. 9 is a functional block diagram showing an example of a functional configuration of an information processing apparatus 10A according to a second embodiment of the disclosed technology. The information processing apparatus 10A according to the second embodiment is different from the information processing apparatus 10 according to the first embodiment in that a comparison processing unit 17 is further provided. The comparison processing unit 17 compares the medical information recorded in the electronic medical record 22 with the related-information associated with the medical information to derive a comparison result.

For example, the comparison processing unit 17 may compare the onset time of the symptom recorded in the record field of the subjective information of the electronic medical record 22 with the onset time of the symptom which is the same as or similar to the symptom specified from the corresponding related-information. For example, in a case in which “have a sense of fatigue since December 10” is recorded in the record field of the subjective information of the electronic medical record 22 and the related-information associated with the information is a message including the description of “listless” transmitted from the user using the messenger application on December 9, the comparison processing unit 17 compares an onset date (December 10) recorded in the electronic medical record 22 with an onset date (December 9) specified from the related-information to derive the comparison result indicating that both onset dates do not match.

Moreover, the comparison processing unit 17 may compare the symptom recorded in the record field of the subjective information of the electronic medical record 22 with the biological information which is the corresponding related-information. For example, in a case in which “have a slight fever since December 10” is recorded in the record field of the subjective information of the electronic medical record 22, the related-information associated with the information is the body temperature measured on December 10, and the measurement value thereof is “37.4° C.”, the symptom (slight fever) recorded in the electronic medical record 22 is compared with the body temperature (37.4° C.) specified from the related-information to derive the comparison result indicating that both the symptom and the body temperature match.

The display processing unit 16 displays the comparison result, which is derived by the comparison processing unit 17, on the display screen 200. FIG. 10 is a diagram showing an example of a display form of the comparison result derived by the comparison processing unit 17. In the example shown in FIG. 10, in a state in which the medical information recorded in the electronic medical record 22 is displayed on the display screen 200, the related-information associated with the medical information is displayed on the display screen 200 together with the comparison result with the medical information by the mouse over pop-up. In this example, since the onset date (December 10) recorded in the electronic medical record 22 and the onset date (December 9) specified from the related-information are different, a message indicating that the content of the related-information and the content of the medical information recorded in the electronic medical record 22 do not match is displayed on the display screen 200.

It should be noted that the display form of the comparison result derived by the comparison processing unit 17 is not limited to the aspect described above. For example, at least one of a color, a size, or a font of a character describing the medical information in the electronic medical record 22 may be different depending on whether the medical information and the related-information match or do not match. Moreover, for example, in a case in which the medical information and the related-information do not match, enhancement display, such as highlighting the character describing the medical information in the electronic medical record 22, may be performed.

With the information processing apparatus 10A according to the present embodiment, it is possible to call attention in a case in which medical information lacking in the accuracy may be recorded in the electronic medical record 22.

In the embodiments described above, for example, as a hardware structure of processing units that execute various pieces of processing, such as the user information acquisition unit 11, the record processing unit 12, the electronic medical record acquisition unit 13, the related-information extraction unit 14, the association processing unit 15, the display processing unit 16, and the comparison processing unit 17, various processors described below can be used. The various processors include, as described above, in addition to the CPU and the GPU, which are general-purpose processors which execute software (program) and function as various processing units, a programmable logic device (PLD) which is a processor whose circuit configuration can be changed after manufacture, such as an FPGA, and a dedicated electric circuit which is a processor having a circuit configuration which is designed for exclusive use in order to execute specific processing, such as an application specific integrated circuit (ASIC).

One processing unit may be composed of one of the various processors or may be composed of a combination of two or more processors of the same type or different types (for example, a combination of a plurality of FPGAs or a combination of a CPU and an FPGA). Moreover, a plurality of the processing units may be composed of one processor.

A first example of the configuration in which the plurality of processing units are composed of one processor is a form in which one processor is composed of a combination of one or more CPUs and software and the processor functions as the plurality of processing units, as represented by the computer, such as a client and a server. Second, as represented by a system on chip (SoC) or the like, there is a form of using a processor that realizes the function of the entire system including the plurality of processing units by one integrated circuit (IC) chip. As described above, various processing units are composed of one or more of the various processors as the hardware structure.

Further, more specifically, an electric circuit (circuitry) obtained by combining circuit elements, such as semiconductor elements, can be used as the hardware structure of the various processors.

Moreover, in the embodiments described above, the aspect has been described in which the information processing program 110 is stored (installed) in advance in the non-volatile memory 103, but the disclosed technology is not limited to this. The information processing program 110 may be provided in a form of being recorded in the recording medium, such as a compact disc read only memory (CD-ROM), a digital versatile disc read only memory (DVD-ROM), and a universal serial bus (USB) memory. Moreover, the information processing program 110 may be provided in a form being downloaded from an external device via a network. That is, the program described in the present embodiment (that is, a program product) may be provided by the recording medium or in a form of being distributed from an external computer.

What is claimed is:

1. An information processing apparatus comprising:
at least one processor,
wherein the processor is configured to:
acquire medical document data in which medical information obtained by a medical examination of a user is recorded,
acquire at least one of input information which is input to a user terminal device used by the user or biological information which is measured for the user,
extract related-information which is related to the medical information from at least one of the input information or the biological information, and
perform association processing of associating the extracted related-information with related medical information.

2. The information processing apparatus according to claim 1,
wherein the processor is configured to
display the medical information on a display screen, and
display the related-information, which is associated with the medical information displayed on the display screen, on the display screen based on an input operation or a selection operation.

3. The information processing apparatus according to claim 2,
wherein the processor is configured to display a result of comparing the medical information with the related-information associated with the medical information on the display screen.

4. The information processing apparatus according to claim 1,
wherein the processor is configured to perform the association processing for information obtained through an interview with the user out of the medical information.

5. The information processing apparatus according to claim 1,
wherein the input information includes a search keyword input to a search engine.

6. The information processing apparatus according to claim 1,
wherein the input information includes a content transmitted or dispatched to a specified or unspecified party.

7. The information processing apparatus according to claim 1,
wherein the processor is configured to
perform processing of selecting input information including a specific word related to a physical condition of the user out of the acquired input information and recording the selected input information in a recording medium, and
perform the association processing for the input information recorded in the recording medium.

8. The information processing apparatus according to claim 1,
wherein the biological information is information measured by a wearable device worn by the user.

9. The information processing apparatus according to claim 1,
wherein the medical document data is an electronic medical record.

10. An information processing method executed by at least one processor provided in an information processing apparatus, the method comprising:
acquiring medical document data in which medical information obtained by a medical examination of a user is recorded;
acquiring at least one of input information which is input to a user terminal device used by the user or biological information which is measured for the user;
extracting related-information which is related to the medical information from at least one of the input information or the biological information; and
associating the extracted related-information with related medical information.

11. A non-transitory computer-readable storage medium storing an information processing program causing at least one processor provided in an information processing apparatus to execute a process comprising:
acquiring medical document data in which medical information obtained by a medical examination of a user is recorded;
acquiring at least one of input information which is input to a user terminal device used by the user or biological information which is measured for the user;
extracting related-information which is related to the medical information from at least one of the input information or the biological information; and
associating the extracted related-information with related medical information.
